(19) United States
(12) Patent Application Publication
(10) Pub. No.: US 2024/0000163 A1
(43) Pub. Date: Jan. 4, 2024

(54) SKID-RESISTANT ELASTIC WAIST SHAPING BELT AND WAIST SHAPING METHOD THEREOF

(71) Applicant: Xiamen Hexin Technology Co., Ltd., Xiamen (CN)

(72) Inventors: Miaoling Zhang, Xiamen (CN); Wenjing Fan, Xiamen (CN); Zhennan Xu, Xiamen (CN)

(21) Appl. No.: 17/875,404

(22) Filed: Jul. 27, 2022

(30) Foreign Application Priority Data
Jul. 1, 2022 (CN) 202210775648.0

(51) Int. Cl. A41C 1/00

(52) U.S. Cl. CPC A41C 1/006

(57) ABSTRACT

A skid-resistant elastic waist shaping belt includes: an elastic belt, which has a shape of a long sheet; a fixing portion, which is provided at one end of the elastic belt and has a width greater than a width of the elastic belt; a first connection element, which is provided at another end of the elastic belt; and a second connection element, which is provided at a position of the elastic belt close to the first connection element. An inner surface of the fixing portion is attached to a human body; the elastic belt is wound for multiple turns along a same circumference from a position connecting to the fixing portion; and the first connection element and the second connection element are connected to bind the waist shaping belt at a waist-abdomen part of the human body.

### CROSS REFERENCE TO RELATED APPLICATIONS

The present application claims the benefit of Chinese Patent Application No. 202210775648.0 filed on Jul. 1, 2022, the contents of which are incorporated herein by reference in their entirety.

### FIELD OF THE INVENTION

The present disclosure relates to the technical filed of body shapers, and in particular, to a skid-resistant elastic waist shaping belt and a waist shaping method thereof.

### BACKGROUND OF THE INVENTION

With the advancement of society and improvements of economic incomes and living standards of people, more and more people start to pay attention to management of their own body shape. It is a nature of a female to pursue beauty, and currently more and more females start to use methods such as wearing a body shaper, performing a surgery, and doing exercise to achieve a better body shape. At present, popular ways of body shaping include: body shaper, Pilates, yoga, liposuction, fitness exercise, and the like, and most females hope that they have a body shape as perfect as possible. Accordingly, losing weight and body shaping are first choices of most females currently. Wearing a body shaper is the most convenient and easiest one among these body-shaping methods, and thus is accepted and favored by most females.

At present, common winding waist shaping belts on the market are generally medical bandages. The medical bandages have no elasticity and poor comfort. Displacement easily occurs because there is no fixing portion as an anchor, and it is not convenient to use the medical bandages, so that effects of stable wearing and abdomen-tightening cannot be achieved. Most of waist shaping belts on the market in recent years do not have a skid-resistant function. During use, the waist shaping belt is easily displaced after being wound, so that the abdomen position often cannot be covered well and flab at the lower abdomen is easily squeezed out. The waist shaping belt curls easily at upper and lower edges easily, deforms when being worn, and has an unsatisfactory waist shaping effect. Therefore, it is necessary to improve the structure of an existing waist shaping belt.

### SUMMARY OF THE INVENTION

Directed against the above problems of a waist shaping belt, such as poor wearing stability, easiness to skid and be displaced when being worn, easiness to curl at upper and lower edges, deformation when being worn, and unsatisfactory waist shaping effect, the disclosure provides a skid-resistant elastic waist shaping belt.

The disclosure discloses a skid-resistant elastic waist shaping belt. The skid-resistant elastic waist shaping belt includes: an elastic belt, which has a shape of a long sheet; a fixing portion, which is provided at one end of the elastic belt and has a width greater than a width of the elastic belt; a first connection element, which is provided at another end of the elastic belt; and a second connection element, which is provided at a position of the elastic belt close to the first connection element. An inner surface of the fixing portion is attached to a human body; the elastic belt is wound for multiple turns along a same circumference from a position connecting to the fixing portion; and the first connection element and the second connection element are connected to bind the waist shaping belt at a waist-abdomen part of the human body.

Further, the fixing portion is provided, at an inner side facing the human body, with a skid-resistant surface.

Further, the elastic belt is provided, on at least one surface, with a skid-resistant layer.

Further, the skid-resistant layer has a diamond-stripe structure, and includes two raised skid-resistant stripes and multiple raised diamond blocks. The skid-resistant stripes are provided along a length direction at edges on both sides of the skid-resistant layer, and the diamond blocks are arranged in an array between the two skid-resistant stripes.

Further, a top surface of each of the skid-resistant stripes is flush with a top surface of each of the diamond blocks.

Further, the skid-resistant stripe includes multiple horizontal stripes and multiple horizontal grooves, and the horizontal stripes and horizontal grooves are sequentially arranged along a length direction of the skid-resistant layer.

Further, the skid-resistant layer has a stripe-groove structure, and includes multiple skid-resistant bands and skid-resistant grooves which are provided sequentially along a length direction of the skid-resistant layer.

Further, the skid-resistant layer has a plush stripe-groove structure, and includes multiple skid-resistant bands and skid-resistant grooves which are provided sequentially along a length direction of the skid-resistant layer. A top surface of each of the skid-resistant bands is provided with a plush layer.

Further, the elastic belt includes 50-75% content of polyurethane fiber (spandex fiber), 10-20% content of polyamide fiber (nylon fiber), and other fibers.

A waist shaping method of a skid-resistant elastic waist shaping belt is provided. The waist shaping method is implemented based on the above skid-resistant elastic waist shaping belt. The skid-resistant elastic waist shaping belt includes: an elastic belt, which has a shape of a long sheet; a fixing portion, which is provided at one end of the elastic belt and has a width greater than a width of the elastic belt; a first connection element, which is provided at another end of the elastic belt; and a second connection element, which is provided at a position of the elastic belt close to the first connection element. An inner side of the fixing portion is attached to a human body; the elastic belt is wound for multiple turns along a same circumference from a position connecting to the fixing portion; and the first connection element and the second connection element are connected to bind the waist shaping belt at a waist-abdomen part of the human body. The waist shaping method includes the following steps:
step 1 of attaching the inner side of the fixing portion to a lower waist-abdomen part of the human body;
step 2 of winding the elastic belt, which is in a tension state, along a same circumference in a partially overlapping manner from an end connecting to the fixing portion as a starting end to another end of the elastic belt; and
step 3 of connecting the first connection element and the second connection element, to achieve tightening of the waist-abdomen part by the waist shaping belt.

The skid-resistant elastic waist shaping belt and the waist shaping method thereof are implemented to have the following beneficial effects:
the skid-resistant elastic waist shaping belt put forward in the disclosure adopts a fixing portion and an elastic belt which have a skid-resistant function. The fixing portion covers abdomen of a human body, is not easy to skid, and has good stability. The elastic belt has a skid-resistant layer, and does not easily curl at edges. The skid-resistant elastic waist shaping belt has a simple design structure on the whole, is easy to wear, and has a good abdomen-tightening effect.

### BRIEF DESCRIPTION OF THE DRAWINGS

In order to better describe embodiments of the disclosure or technical solutions in existing technologies, drawings required in describing the embodiments and the existing technologies will be described briefly below. Obviously, drawings described below are only drawings of some embodiments of the disclosure, and those of ordinary skills in the art can also obtain other drawings based on these drawings without making any creative effort.

FIG. 1 schematically shows a structure of a skid-resistant elastic waist shaping belt according to an embodiment of the disclosure;

FIG. 2 schematically shows a skid-resistant elastic waist shaping belt in a spreading state according to an embodiment of the disclosure;

FIG. 3 schematically shows an exploded structure of a fixing portion of a skid-resistant elastic waist shaping belt according to an embodiment of the disclosure;

FIG. 4 schematically shows a partial structure of a skid-resistant layer of an elastic belt of a skid-resistant elastic waist shaping belt according to embodiment one of the disclosure;

FIG. 5 schematically shows the skid-resistant elastic waist shaping belt during use according to embodiment one of the disclosure;

FIG. 6 schematically shows a partial structure of a skid-resistant layer of an elastic belt of a skid-resistant elastic waist shaping belt according to embodiment two of the disclosure;

FIG. 7 schematically shows a partial structure of a skid-resistant layer of an elastic belt of a skid-resistant elastic waist shaping belt according to embodiment three of the disclosure; and

FIG. 8 schematically shows a partial structure of a skid-resistant layer of an elastic belt of a skid-resistant elastic waist shaping belt according to embodiment four of the disclosure.

In the drawings, reference numerals are as follows: elastic belt—1; fixing portion—2; first connection element 3; second connection element—4; skid—resistant surface—5; skid-resistant layer—6; skid-resistant stripe—61; horizontal stripe—611; horizontal groove—612; diamond block—62; skid-resistant band—(63; 65); skid-resistant groove—(64; 66); and plush layer—67.

### DETAILED DESCRIPTION OF THE EMBODIMENTS

Technical solutions in embodiments of the disclosure will be described clearly and completely with reference to the accompanying drawings in the embodiments. Obviously, the embodiments described are only some embodiments, rather than all embodiments, of the disclosure. All other embodiments obtained by those of ordinary skills in the art based on the embodiments of the disclosure without making any creative effort fall into the protection scope of the disclosure.

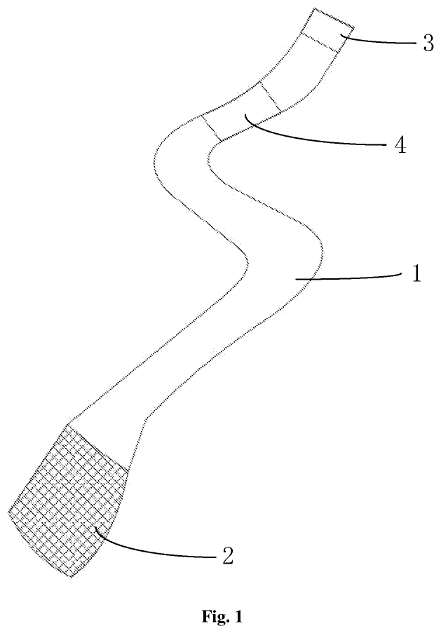

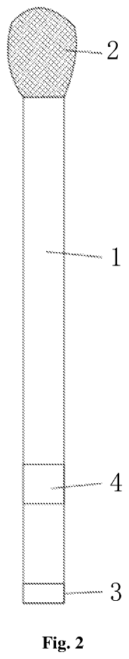

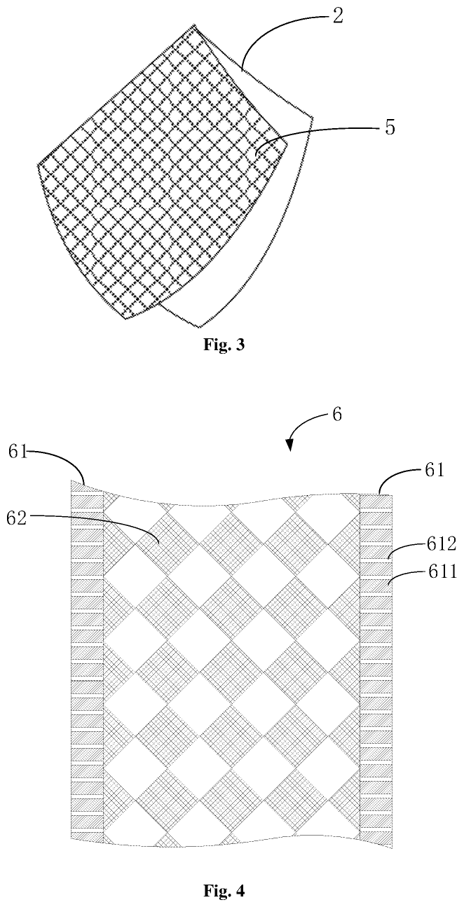

Please refer to FIG. 1, FIG. 2, and FIG. 3, according to embodiment one of the disclosure, an elastic belt 1 is included, and the elastic belt 1 has a shape of a long sheet and has elasticity. A fixing portion 2 is provided at one end of the elastic belt 1, and the fixing portion 2 has a width greater than a width of the elastic belt 1 and has a length far less than a length of the elastic belt 1. The fixing portion 2 is provided at an inner side surface with a skid-resistant surface 5, and the skid-resistant surface 5 has a looped pile fabric structure, which has good comfort and a great skid-resistant effect.

A first connection element 3 is provided at another end of the elastic belt 1 distant from the fixing portion 2. A second connection element 4 is provided at a position of the elastic belt 1 close to the first connection element 3. The skid-resistant surface 5 of the fixing portion 2 is attached to abdomen of a human body. The elastic belt 1 is wound for multiple turns along a same circumference from a position connecting to the fixing portion 2. The first connection element 3 and the second connection element 4 are connected to bind a waist shaping belt at a waist-abdomen part of the human body, and the first connection element 3 and the second connection element 4 are connected using Velcro.

Please refer to FIG. 4, the elastic belt 1 is provided thereon with a skid-resistant layer 6, and the skid-resistant layer 6 is located at the same side as the skid-resistant surface 5 of the fixing portion 2. That is, when the waist shaping belt is being worn, the skid-resistant layer 6 of the elastic belt 1 faces the human body. It is certain that a skid-resistant layer 6 may also be provided on another side surface of the elastic belt 1, to strengthen a skid-resistant effect.

The skid-resistant layer 6 has a diamond-stripe structure, and includes two raised skid-resistant stripes 61 and multiple raised diamond blocks 62. A top surface of each of the skid-resistant stripes 61 is flush with a top surface of each of the diamond blocks 62. The skid-resistant stripes 61 are provided along a length direction at edges on both sides of the skid-resistant layer 6, and the diamond blocks 62 are arranged in an array between the two skid-resistant stripes 61.

Specifically, a vertex at one of four corners of a diamond block is connected to a vertex at one of four corners of an adjacent diamond block to form an array arrangement. The diamond block 62 is provided at an upper surface with a texture surface, to provide a better skid-resistant effect.

The skid-resistant stripe 61 includes multiple horizontal stripes 611 and multiple horizontal grooves 612, and the horizontal stripes 611 and horizontal grooves 612 are sequentially arranged along a length direction of the skid-resistant layer 6. The skid-resistant effect of the skid-resistant layer 6 is enhanced by arranging the horizontal stripes 611 and horizontal grooves 612. In addition, a thickness of both edges of the elastic belt 1 is increased. Therefore, the elastic belt 1 does not easily curl at the edges, and does not deform easily when the waist shaping belt is being worn, so that a waist shaping effect is not affected.

It should be noted that, an inner surface of a waist shaping belt in existing technologies does not have a skid-resistant effect, so that the waist shaping belt easily skids along the body when being worn and has poor stability. However, the waist shaping belt according to the disclosure not only has a good elastic function but also has a good skid-resistant function.

Components of the elastic belt 1 in the present embodiment include 50-75% of polyurethane fiber, 10-20% of polyamide fiber, and other fibers.

The elastic belt with a high content of polyurethane fiber components has a long service life and provides a good body feeling.

The polyamide fiber has a heat conductivity coefficient of 0.244-0.337, and a fabric of the polyamide fiber provides a cool body feeling. The polyamide fiber has features of a cool feeling fiber. The cool feeling fiber is a fiber which has fast heat conduction, a cool feeling of contact, a large application heat capacity, and a high coefficient of heat conduction, or is a fiber which achieves heat transfer by the fiber and a cool feeling of contact by an addition of a medium with such properties into the fiber and a combination of different fibers. Application of the cool feeling fiber varies according to implementation manners and application fields. A mode of “polyamide+viscose” popular on the market has an obvious cool feeling effect, and is mainly used for bed sheets, quilts, and the like used in summer. Viscose is usually called ice silk. This is mainly because viscose is smooth, has a drape feeling, and absorbs moisture, and a filament yarn fabric of the viscose provides a cool feeling to a person when being worn. Therefore, after the polyamide and the viscose are combined, the cool feeling is more obvious.

The polyamide fiber may be replaced by a polyester fiber. The polyester fiber has a heat conductivity coefficient of 0.084, which is less than the heat conductivity coefficient of the polyamide fabric, and thus provides a warm body feeling.

Other fibers include an antibacterial deodorant fiber. The antibacterial deodorant fiber refers to a fiber which has an antibacterial and bacteriostatic effect. An antibacterial fiber mainly includes two types. One type of antibacterial fiber is a fiber which itself has an antibacterial and bacteriostatic effect, such as hemp, apocynum, chitin fiber, metal fiber, and the like. Another type of antibacterial fiber is an antibacterial fiber formed by adding an antibacterial agent into the fiber during chemical fiber spinning or modification by means of the chelation technology, the nanotechnology, the powder addition technology, and the like.

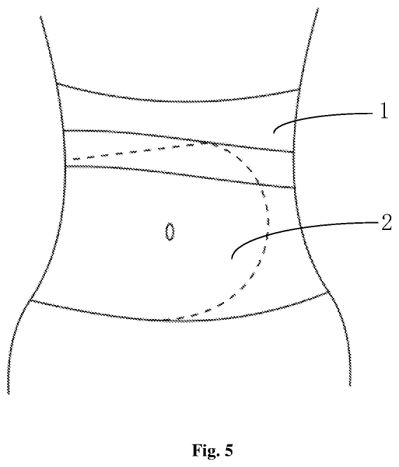

Please refer to FIG. 1 and FIG. 5, the disclosure puts forward a waist shaping method of a skid-resistant elastic waist shaping belt, and the method is implemented based on the above skid-resistant elastic waist shaping belt. The skid-resistant elastic waist shaping belt includes: an elastic belt 1, which has a shape of a long sheet; a fixing portion 2, which is provided at one end of the elastic belt 1 and has a width greater than a width of the elastic belt 1; a first connection element 3, which is provided at another end of the elastic belt 1; and a second connection element 4, which is provided at a position of the elastic belt 1 close to the first connection element 3. An inner side of the fixing portion 2 is attached to a human body. The elastic belt 1 is wound for multiple turns along a same circumference from a position connecting to the fixing portion 2. The first connection element 3 and the second connection element 4 are connected to bind the waist shaping belt at a waist-abdomen part of the human body. The waist shaping method includes the following steps:
step 1 of attaching the inner side of the fixing portion to a lower waist-abdomen part of the human body;
step 2 of winding the elastic belt, which is in a tension state, along a same circumference in a partially overlapping manner from an end connecting to the fixing portion as a starting end to another end of the elastic belt; and
step 3 of connecting the first connection element and the second connection element, to achieve tightening of the waist-abdomen part by the waist shaping belt.

The skid-resistant elastic waist shaping belt and the waist shaping method thereof are implemented to have the following beneficial effects:
the skid-resistant elastic waist shaping belt put forward in the disclosure adopts a fixing portion and an elastic belt which have a skid-resistant function. The fixing portion covers abdomen of a human body, is not easy to skid, and has good stability. The elastic belt has a skid-resistant layer, and does not easily curl at edges. The skid-resistant elastic waist shaping belt has a simple design structure on the whole, is easy to wear, and has a good abdomen-tightening effect.

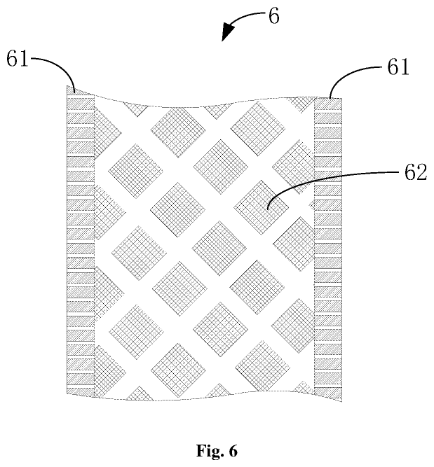

Please refer to FIG. 1 and FIG. 6, according to embodiment two of the disclosure, an elastic belt 1 is included, and the elastic belt 1 has a shape of a long sheet and has elasticity. A fixing portion 2 is provided at one end of the elastic belt 1, and the fixing portion 2 has a width greater than a width of the elastic belt 1 and has a length far less than a length of the elastic belt 1. A first connection element 3 is provided at another end of the elastic belt 1 distant from the fixing portion 2. A second connection element 4 is provided at a position of the elastic belt 1 close to the first connection element 3. A skid-resistant surface 5 of the fixing portion 2 is attached to abdomen of a human body. The elastic belt 1 is wound for multiple turns along a same circumference from a position connecting to the fixing portion 2. The first connection element 3 and the second connection element 4 are connected to bind a waist shaping belt at a waist-abdomen part of the human body, and the first connection element 3 and the second connection element 4 are connected using multiple rows of hooks and loops.

The elastic belt 1 is provided thereon with a skid-resistant layer 6, and the skid-resistant layer 6 of the elastic belt 1 faces the human body. The skid-resistant layer 6 has a diamond-stripe structure, and includes two raised skid-resistant stripes 61 and multiple raised diamond blocks 62. A top surface of each of the skid-resistant stripes 61 is flush with a top surface of each of the diamond blocks 62. The skid-resistant stripes 61 are provided along a length direction at edges on both sides of the skid-resistant layer 6, and the diamond blocks 62 are arranged in an array between the two skid-resistant stripes 61. The diamond blocks 62 are spaced by stripe-shaped grooves provided among them. An upper surface of each of the diamond blocks 62 and an upper surface of each of the skid-resistant stripes 61 are provided with a texture surface, to provide a better skid-resistant effect.

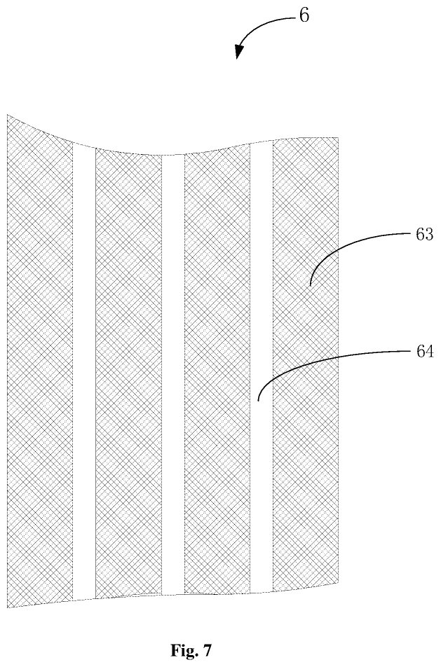

Please refer to FIG. 1 and FIG. 7, according to embodiment three of the disclosure, an elastic belt 1 is included, and the elastic belt 1 has a shape of a long sheet and has elasticity. A fixing portion 2 is provided at one end of the elastic belt 1, and the fixing portion 2 has a width greater than a width of the elastic belt 1 and has a length far less than a length of the elastic belt 1. A first connection element 3 is provided at another end of the elastic belt 1 distant from the fixing portion 2. A second connection element 4 is provided at a position of the elastic belt 1 close to the first connection element 3. A skid-resistant surface 5 of the fixing portion 2 is attached to abdomen of a human body. The elastic belt 1 is wound for multiple turns along a same circumference from a position connecting to the fixing portion 2. The first connection element 3 and the second connection element 4 are connected to bind a waist shaping belt at a waist-abdomen part of the human body, and the first connection element 3 and the second connection element 4 are connected using multiple rows of hooks and loops.

The elastic belt 1 is provided thereon with a skid-resistant layer 6, and the skid-resistant layer 6 of the elastic belt 1 faces the human body. The skid-resistant layer 6 has a stripe-groove structure, and includes multiple skid-resistant bands 63 and skid-resistant grooves 64 which are provided sequentially along a length direction of the skid-resistant layer 6. Specifically, a skid-resistant band 63 is provided at each of edges on both sides of the skid-resistant layer 6, and then the skid-resistant grooves 64 and the skid-resistant bands 63 are provided sequentially between the skid-resistant bands 63 which are provided at the edges on both sides of the skid-resistant layer 6. The skid-resistant bands 63 and the skid-resistant grooves 64 have a length the same as a length of the skid-resistant layer 6. An upper surface of each of the skid-resistant bands 63 is provided with a texture surface, to provide a better skid-resistant effect.

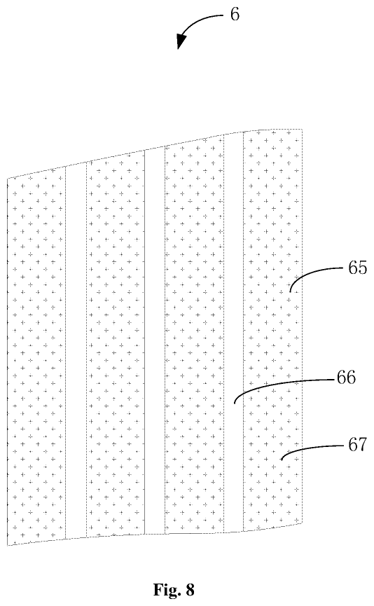

Please refer to FIG. 1 and FIG. 8, according to embodiment four of the disclosure, an elastic belt 1 is included, and the elastic belt 1 has a shape of a long sheet and has elasticity. A fixing portion 2 is provided at one end of the elastic belt 1, and the fixing portion 2 has a width greater than a width of the elastic belt 1 and has a length far less than a length of the elastic belt 1. A first connection element 3 is provided at another end of the elastic belt 1 distant from the fixing portion 2. A second connection element 4 is provided at a position of the elastic belt 1 close to the first connection element 3. A skid-resistant surface 5 of the fixing portion 2 is attached to abdomen of a human body. The elastic belt 1 is wound for multiple turns along a same circumference from a position connecting to the fixing portion 2. The first connection element 3 and the second connection element 4 are connected to bind a waist shaping belt at a waist-abdomen part of the human body, and the first connection element 3 and the second connection element 4 are connected using multiple rows of hooks and loops.

The elastic belt 1 is provided thereon with a skid-resistant layer 6, and the skid-resistant layer 6 of the elastic belt 1 faces the human body. The skid-resistant layer 6 has a plush stripe-groove structure, and includes multiple skid-resistant bands 65 and skid-resistant grooves 66 which are provided sequentially along a length direction of the skid-resistant layer 6. A top surface of each of the skid-resistant bands 65 is provided with a plush layer 67, which provides a better comfortable feeling when contacting with the human body.

The above described embodiments are merely preferred embodiments of the disclosure and are not intended for limiting the disclosure. Modifications, equivalent substitutions, and improvements made within the spirit and principles of the disclosure shall all fall within the protection scope of the present disclosure.

1. A skid-resistant elastic waist shaping belt, comprising: an elastic belt (1), which has a shape of a long sheet; a fixing portion (2), which is provided at one end of the elastic belt (1) and has a width greater than a width of the elastic belt (1); a first connection element (3), which is provided at another end of the elastic belt (1); and a second connection element (4), which is provided at a position of the elastic belt (1) close to the first connection element (3), wherein an inner surface of the fixing portion (2) is attached to a human body; the elastic belt (1) is wound for multiple turns along a same circumference from a position connecting to the fixing portion (2); and the first connection element (3) and the second connection element (4) are connected to bind the waist shaping belt at a waist-abdomen part of the human body.

2. The skid-resistant elastic waist shaping belt according to claim 1, wherein the fixing portion (2) is provided, at an inner side facing the human body, with a skid-resistant surface (5).

3. The skid-resistant elastic waist shaping belt according to claim 2, wherein the elastic belt (1) is provided, on at least one surface, with a skid-resistant layer (6).

4. The skid-resistant elastic waist shaping belt according to claim 3, wherein the skid-resistant layer (6) has a diamond-stripe structure, and comprises two raised skid-resistant stripes (61) and multiple raised diamond blocks (62), wherein the skid-resistant stripes (61) are provided along a length direction at edges on both sides of the skid-resistant layer (6), and the diamond blocks (62) are arranged in an array between the two skid-resistant stripes (61).

5. The skid-resistant elastic waist shaping belt according to claim 4, wherein a top surface of each of the skid-resistant stripes (61) is flush with a top surface of each of the diamond blocks (62).

6. The skid-resistant elastic waist shaping belt according to claim 5, wherein the skid-resistant stripe (61) comprises multiple horizontal stripes (611) and multiple horizontal grooves (612), and the horizontal stripes (611) and horizontal grooves (612) are sequentially arranged along a length direction of the skid-resistant layer (6).

7. The skid-resistant elastic waist shaping belt according to claim 3, wherein the skid-resistant layer (6) has a stripe-groove structure, and comprises multiple skid-resistant bands (63) and skid-resistant grooves (64) which are provided sequentially along a length direction of the skid-resistant layer (6).

8. The skid-resistant elastic waist shaping belt according to claim 3, wherein the skid-resistant layer (6) has a plush stripe-groove structure, and comprises multiple skid-resistant bands (65) and skid-resistant grooves (66) which are provided sequentially along a length direction of the skid-resistant layer, wherein a top surface of each of the skid-resistant bands (65) is provided with a plush layer (67).

9. The skid-resistant elastic waist shaping belt according to claim 1, wherein the elastic belt (1) comprises 50-75% of spandex fiber, 10-20% of nylon fiber, and other fibers.

10. A waist shaping method of a skid-resistant elastic waist shaping belt, wherein the waist shaping method is implemented based on the above skid-resistant elastic waist shaping belt, wherein the skid-resistant elastic waist shaping belt comprises: an elastic belt (1), which has a shape of a long sheet; a fixing portion (2), which is provided at one end of the elastic belt (1) and has a width greater than a width of the elastic belt (1); a first connection element (3), which is provided at another end of the elastic belt (1); and a second connection element (4), which is provided at a position of the elastic belt (1) close to the first connection element (3), wherein an inner side of the fixing portion (2) is attached to a human body; the elastic belt (1) is wound for multiple turns along a same circumference from a position connecting to the fixing portion (2); and the first connection element (3) and the second connection element (4) are connected to bind the waist shaping belt at a waist-abdomen part of the human body, and wherein the waist shaping method comprises the following steps:
step 1 of attaching the inner side of the fixing portion to a lower waist-abdomen part of the human body;
step 2 of winding the elastic belt, which is in a tension state, along a same circumference in a partially overlapping manner from an end connecting to the fixing portion as a starting end to another end of the elastic belt; and
step 3 of connecting the first connection element and the second connection element, to achieve tightening of the waist-abdomen part by the waist shaping belt.
